(19) United States
(12) Patent Application Publication
(10) Pub. No.: US 2023/0359276 A1
(43) Pub. Date: Nov. 9, 2023

(54) Tissue-Integrating Neural Interfaces

(71) Applicant: Massachusetts Institute of Technology, Cambridge, MA (US)

(72) Inventors: Anthony Tabet, Woburn, MA (US); Polina Olegovna ANIKEEVA, Lexington, MA (US)

(21) Appl. No.: 18/314,317

(22) Filed: May 9, 2023

Related U.S. Application Data
(60) Provisional application No. 63/339,654, filed on May 9, 2022.

(51) Int. Cl. A61B 5/00; G06F 3/01

(52) U.S. Cl. CPC G06F 3/015; A61B 5/6868

(57) ABSTRACT

Solvent evaporation or entrapment-driven (SEED) integration is a rapid, robust, and modular approach to creating multifunctional fiber-based neural interfaces. SEED integration brings together electrical, optical, and microfluidic modalities within a co-polymer comprised of watersoluble poly(ethylene glycol) tethered to water-insoluble poly(urethane) (PU-PEG). The resulting neural interfaces can perform optogenetics and electrophysiology simultaneously. They can also be used to deliver cellular cargo with high viability. Upon exposure to water, PU-PEG cladding spontaneously forms a hydrogel, which, in addition to enabling integration of modalities, can harbor small molecules and nanomaterials that can be released into local tissue following implantation. For example, the hydrogel of a SEED-integrated neural interface can host a custom nanodroplet-forming block polymer for delivery of hydrophobic small molecules in vitro and in vivo. SEED integration widens the chemical toolbox and expands the capabilities of multifunctional neural interfaces.

### CROSS REFERENCE TO RELATED APPLICATION(S)

This application claims the priority benefit, under 35 U.S.C. 119(e), of U.S. Application No. 63/339,654, filed May 9, 2022, which is incorporated herein by reference in its entirety for all purposes.

### BACKGROUND

The fiber drawing process enables the fabrication of flexible neural probes that can simultaneously interrogate neuronal circuits via electrical, optical, and chemical modalities. During fiber drawing, a macroscopic model (the preform) of the desired probe is fabricated and drawn into hundreds of meters of fibers with microscale features. To date, these probes have enabled one-step optogenetics, in vivo photopharmacology, and in-situ electrochemical synthesis of gaseous molecules for neuromodulation.

Despite these advancements, this fiber drawing approach has several limitations. To be co-drawable, the constituent materials should have similar glass transition temperatures (for polymers) and melting temperatures (for metals). The resulting melt viscosities should also be compatible to obtain stable draw conditions for maintaining the cross-sectional geometry of the preform. Additionally, while thermal drawing yields hundreds of meters of fiber at once, that fiber is cut into individual centimeter-long devices, each of which is manually connected to back-end hardware, a laborious process that is a fabrication bottleneck. Furthermore, the polymer cladding of these fibers serves only passive structural or electrical insulation purposes, significantly adding to the device footprint with little added functionality.

### SUMMARY

Hydrogels are an attractive class of materials for neural interfaces. The mammalian brain itself is a weak hydrogel with a complex modulus G* on the order of 1 kPa. While hydrogels alone can serve as neural interfaces, for example, as optical waveguides or electrodes, their use in multifunctional neural probes has been more limited. Additionally, while hydrogels have been extensively used as depots for sustained release of bioactive molecules, this drug delivery capability has not yet been extended to multifunctional neural interfaces.

Here, we disclose multifunctional, hydrogel-based, tissue-integrating neural interfaces that can be loaded with and elute drugs and/or nanomaterials. These neural interfaces can be made using thermal drawing with a solvent evaporation or entrapment-driven (SEED) integration process. For example, an inventive neural interface can be made by forming a fiber bundle from a plurality of fibers, at least partially coating the fiber bundle in a layer of poly(urethane)-poly(ethylene glycol) (PU-PEG), and at least partially coating the layer of PU-PEG in a layer of hydrogel.

The plurality of fibers can include at least one of an optical fiber, an electrical fiber, or a microfluidic fiber; for example, it might include an optical fiber, an electrical fiber, and a microfluidic fiber.

Coating the fiber bundle in the layer of PU-PEG may include dipping the fiber bundle in a solution of PU-PEG and drying the solution of PU-PEG on the fiber bundle. Similarly, coating the layer of PU-PEG in the layer of hydrogel may include dipping the fiber bundle in a hydrogel bath after forming the layer of PU-PEG on the fiber bundle. The layer of hydrogel can include at least one of a protein, glycan, synthetic polymer, biopolymer, gelatin, laminin, hyaluronic acid, alginate, or Matrigel.

If desired, the layer of hydrogel can be loaded with a molecule configured to interact with and/or affect a human brain. The layer of hydrogel can be loaded with at least one of a hydrophobic molecule, a hydrophilic molecule, a peptide, or a protein. Hydrophilic molecules, peptides, and/or proteins can simply be mixed with the hydrogel precursor solutions. The layer of hydrogel can also be loaded with cells.

An inventive neural interface may include a fiber bundle comprising a plurality of fibers, a layer of PU-PEG at least partially surrounding the fiber bundle, and a layer of hydrogel at least partially surrounding the layer of PU-PEG.

All combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. The terminology explicitly employed herein that also may appear in any disclosure incorporated by reference should be accorded a meaning most consistent with the particular concepts disclosed herein.

### BRIEF DESCRIPTIONS OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar components).

FIG. 1A illustrates a thermal fiber drawing process for turning a preform into an electronic, optical, or microfluidic fiber for a tissue-integrating neural interface.

FIG. 1B shows three separate, fully connectorized modules made with electronic, optical, and microfluidic fibers, respectively, (top) and the chemical structure of the integrated poly(urethane)-poly(ethylene glycol) (PU-PEG) hydrogel (bottom) for a tissue-integrating neural interface.

FIG. 1C illustrates solvent evaporation or entrapment-driven (SEED) integration for creating a multifunctional, hydrogel, tissue-integrating neural interface from connectorized modules and PU-PEG.

FIG. 1D shows a stepper motor stage used to control the dip-coating process during SEED integration.

FIG. 1E illustrates additional collagen dipping and curing steps that can be performed as part of SEED integration.

FIG. 1F illustrates SEED integration of a multifunctional fiber.

FIG. 1G shows various components (top) of the assembled PU-PEG hydrogel and cross sections (bottom) of each component and the entire tissue-integrating neural interface.

FIG. 2A shows an awake transgenic Thy1-ChR2 mouse chronically implanted with a hydrogel neural interface (top) and optogenetically invoking action potentials in anesthetized transgenic Thy1-ChR2 mouse with a hydrogel neural interface (bottom).

FIG. 2B shows chronic recordings of optogenetically invoked action potentials in the nucleus accumbens (NAc) in a Thy1-ChR2 mouse acquired with an implanted hydrogel neural interface.

FIG. 2C illustrates the release profile of drugs injected through the microfluidic channel of a neural interface implanted in a Thy1-ChR2 mouse brain using Evans blue dye.

FIG. 2D shows flow cytometry data demonstrating the capability of an inventive neural interface to deliver cells with high viability.

FIG. 3A illustrates a hydrogel neural interface loaded with fluorescein implanted into a 0.6% agarose phantom brain over time.

FIG. 3B is a plot of photoluminescence emitted by fluorescein released from integrated fibers with and without PU-PEG versus time.

FIG. 3C shows confocal microscope images of sections of the brain of a Thy1-ChR2 mouse 72 hours after implantation of a neural interface coated with hydrogel loaded with Evans blue dye into the NAc.

FIG. 3D shows confocal microscope images of sections of the brain of another Thy1-ChR2 mouse 72 hours after implantation of a neural interface coated with hydrogel loaded with Evans blue dye and yellow fluorescent protein (YFP) into the NAc.

FIG. 3E illustrates finite element modeling of mass transport of a small molecule from either a microfluidic channel (left) or hydrogel (right) of a tissue-integrating neural interface 10 minutes after completion of the 3 µL injection (same time point in both cases).

FIG. 3F illustrates both microfluidic and hydrogel-based drug delivery and the resulting convection versus diffusion driving forces, respectively.

FIG. 4 shows the chemical structure of customized polyacetal PA11, a block copolymer, that self-emulsifies into nanodroplets capable of delivering hydrophobic small molecule drugs (bottom).

FIG. 5A is a plot of dynamic light scattering (DLS) data of solutions of PA11 eluted from a PA11/PU-PEG-based fiber and a thick PA11/PU-PEG film.

FIG. 5B shows a transmission electron microscopy (TEM) image of PA11 nanodroplets.

FIG. 5C shows supernatants from films of PU-PEG loaded with Nile Red without (top) or with (bottom) PA11.

FIG. 5D is a bar chart showing photoluminescence of saline solution and Nile Red (NR) loaded into hydrogel neural interfaces with and without PA11.

FIG. 5E is a plot of full fluorescence spectra of the fiber supernatant when both Nile Red and PA11 are incorporated into the hydrogel.

FIG. 5F shows confocal micrographs (60×) of primary rat dorsal root ganglion neurons (DRGs) co-incubated with media containing both PA11 and Nile Red for 24 hours.

FIG. 5G is a bar chart showing photoluminescence for neural interfaces loaded with Nile Red with and without PA11.

FIG. 5H shows elution of NR/PA11 from the hydrogel coating of a neural interface in the NAc of C57BL/6 mice after 72 hours at 4× magnification.

FIG. 5I shows elution of NR/PA11 from the hydrogel coating of a neural interface in the NAc of C57BL/6 mice after 72 hours at 60× magnification.

FIG. 6A is a plot of transmission versus length for CK-10 (upper trace) and polycarbonate (PC)/polymethyl methacrylate (PMMA) optical fibers in neural interfaces.

FIG. 6B is a plot of attenuation per unit length for different optical fibers.

FIG. 7A is a plot of impedance versus frequency for tungsten (upper trace) and carbon nanotube (CNT; lower trace) electrical fibers in neural interfaces.

FIG. 7B is a plot of current versus voltage for CNT and tungsten electrical fibers in neural interfaces.

FIG. 7C is a plot of potential relative to a reference potential versus time for CNTs at different currents, illustrating their utility in electrical stimulation within these multifunctional fibers.

FIG. 7D is a plot of the water window potential limit for CNTs versus tungsten, demonstrating that CNTs maintain their electrical stimulation capabilities within these multifunctional fibers.

FIG. 8 is a plot of change in fluorescence versus time obtained with an implanted neural interface.

FIG. 9A is a plot of change fluorescence versus time obtained with an implanted neural interface at different current levels for electrical stimulation delivered with the implanted neural interface.

FIG. 9B is a plot of change fluorescence versus time obtained with an implanted neural interface at different frequencies of electrical stimulation delivered with the implanted neural interface.

FIG. 10 shows traces of electrical activity evoked and recorded with a chronically implanted neural interface.

FIG. 11 shows peak-to-peak variation of the traces in FIG. 10 over time.

FIG. 12 shows a section of a mouse brain after removal of a chronically implanted hydrogel neural interface.

FIG. 13A illustrates a process for implanting neural interfaces in inoculated flank tumors.

FIG. 13B is a plot of force versus time for detaching a SEED-integrated hydrogel neural interface (solid trace) and a conventional polymer fiber (dashed trace) adhered to skin with a bioadhesive.

FIG. 13C is a bar chart of maximum force for detaching a SEED-integrated hydrogel neural interface (solid trace) and a conventional polymer fiber (dashed trace) adhered to skin with a bioadhesive.

FIG. 13D is a plot of in vivo calcium activity (fluorescence intensity) versus time obtained with an implanted neural interface before and during electrical disruption of melanoma with the implanted neural interface.

FIG. 14 shows traces of fluorescence from cancer cells recorded in vivo by chronically implanted hydrogel neural interfaces.

FIG. 15 shows plots of change in fluorescence versus time for an implanted neural interface delivering KCL (upper trace) and PBS (lower trace) in vivo.

### DETAILED DESCRIPTION

Multifunctional neural interfaces provide a way to interface with the brain electrically, optically, and/or chemically. One goal of these devices, which are made of soft and inert polymers, is to avoid a large inflammatory scar like those associated with steel, silicon, and glass-based devices. To date, many of these neural interfaces have been just plastic, not solvated and not penetrable by cells and tissue.

Other inert neural interfaces include hydrogel-based devices. These hydrogels are also not penetrable by cells and tissue. They are ‘invisible’ in so far as they do not interact with or modulate their surrounding tissue.

In contrast, inventive tissue-integrating neural interfaces can be implanted in the brain without causing large inflammatory scars and can interact with and/or modulate surrounding tissue once implanted. These neural interfaces are fiber-based and can be created using a layer-by-layer approach that yields a hydrogel-coated neural interface with highly customizable surface chemistry. For example, the neural interface can be a brain-integrating device integrated with collagen and/or other materials. Cells can penetrate and dynamically interact with brain-integrating neural interfaces, for example, at or via the collagen surface.

Inventive neural interfaces are made using a solvent evaporation or entrapment-driven (SEED) integration process utilizing PU-PEG hydrogels. SEED integration is a robust and translatable method that is not labor intensive. It does not use oxygen-sensitive chemistry or neurotoxic radicals and takes only a few minutes of active fabrication time. Additionally, devices fabricated with SEED integration can feature active cladding which can be co-loaded with both small molecule drugs and drug nanocarriers for delivery in vivo. These properties make modular hydrogel neural interfaces well suited for fundamental and translational biological research.

Leveraging a SEED integration approach that employs amphiphilic co-polymers makes it possible to create modular hydrogel neural interfaces capable of optogenetics, electrophysiology, and/or microfluidic delivery. These neural interfaces can deliver a variety of cargo, including cellular therapies with a high viability at fast injection rates. Loading model drugs or nanomaterials into a neural interface’s hydrogel enables a separate drug delivery modality with a unique driving force and release profile. Neural interfaces can even deliver hydrophobic cargo, such as hydrophobic small-molecule drugs.

### Solvent Evaporation or Entrapment-Driven (SEED) Integration

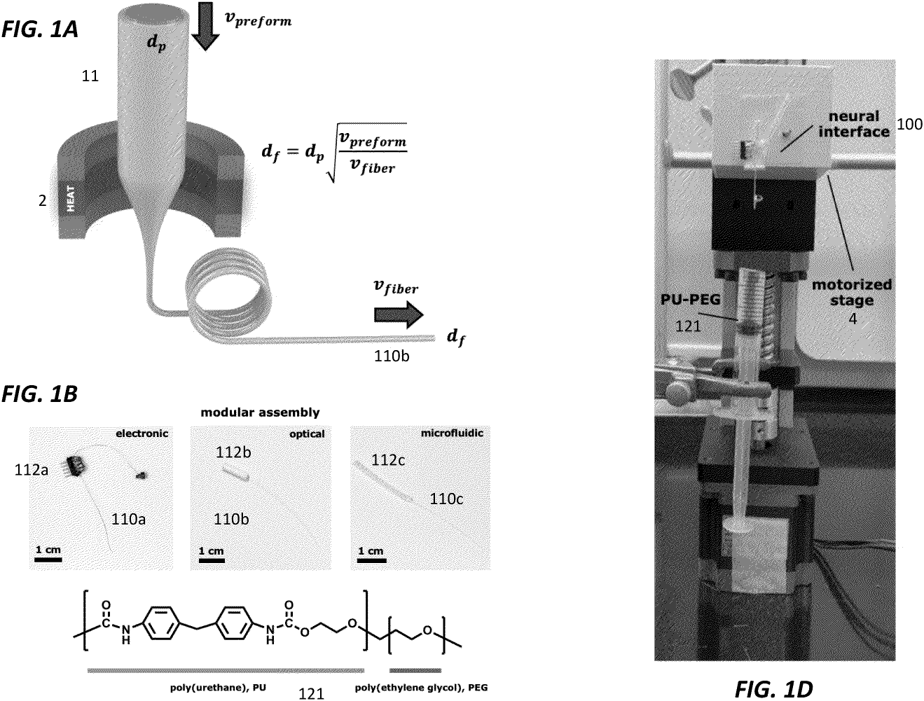

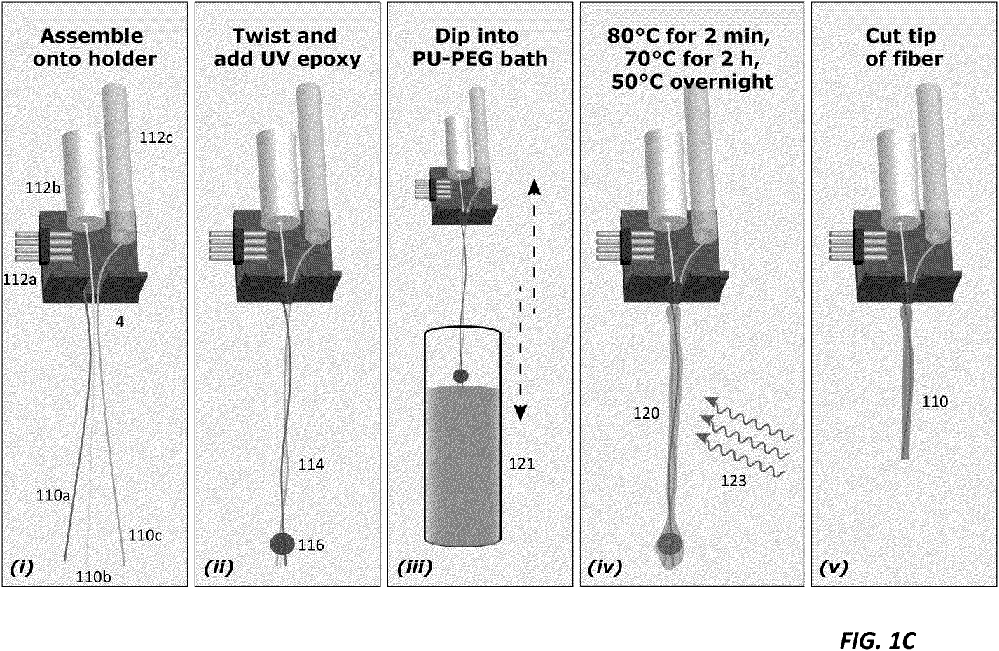

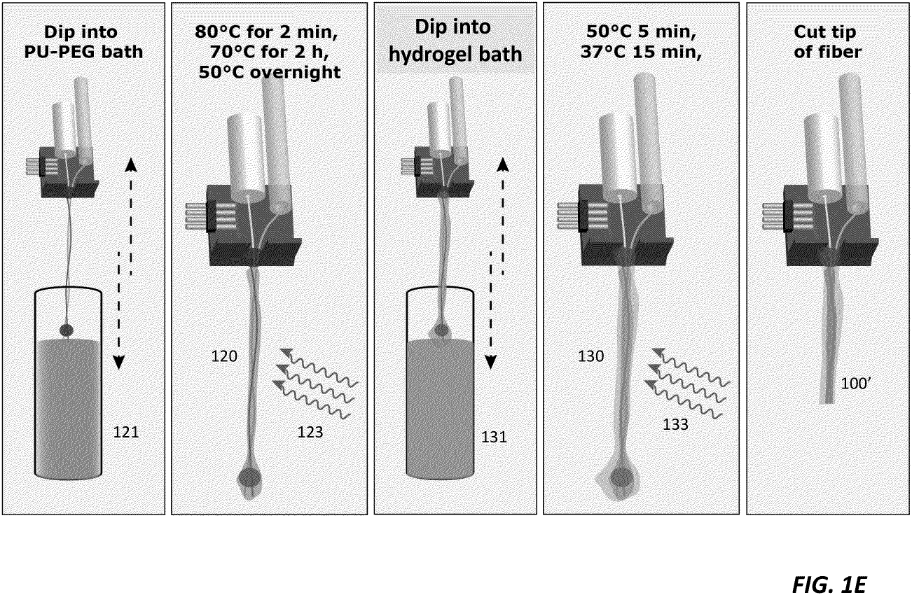

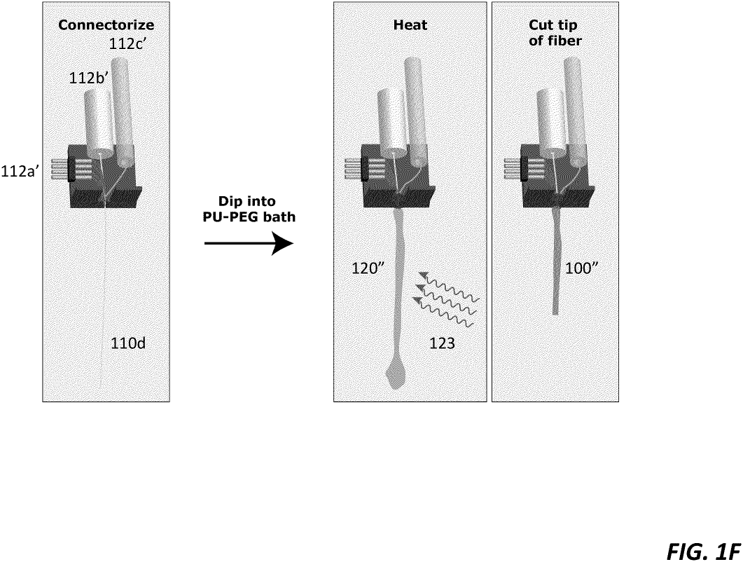

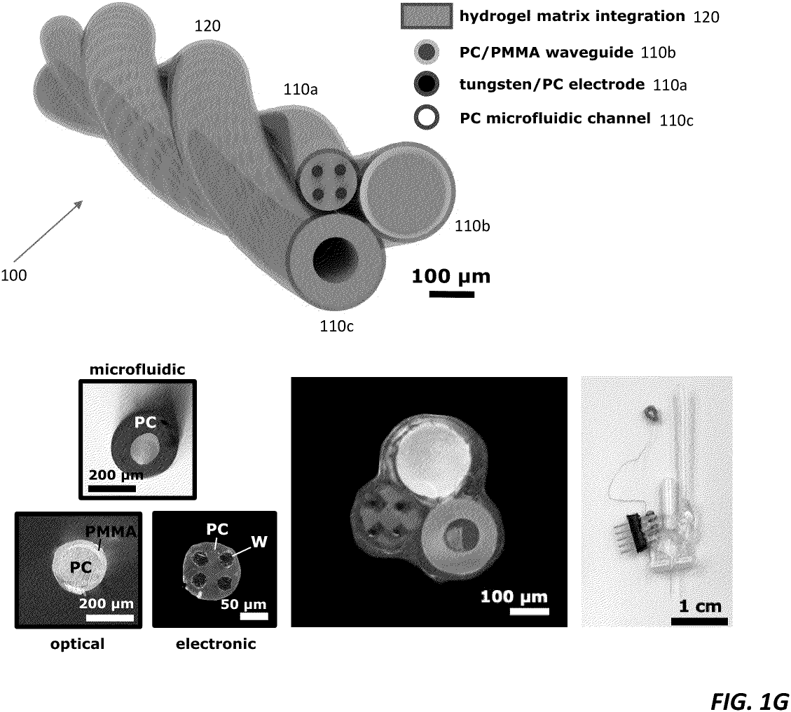

FIGS. 1A-1G illustrate tissue-integrating neural interfaces 100 and how to make them. FIG. 1A illustrates a thermal drawing process for making electrical fibers 110a, optical fibers 110b (shown in FIG. 1A), and/or microfluidic fibers 110c that can be bundled together in the neural interfaces 100. An optical fiber may include a high-index core surrounding by a lower-index cladding for guiding light; an electrical fiber may include one or more conductors (e.g., tungsten wires with diameters of microns) for measuring or applying electrical signals; and a microfluidic fiber may be a fiber with a hollow channel or core for conveying liquid from end to another. For instance, an electrical fiber 110a may be a recording electrode array fiber with four 25 µm tungsten (W) wires within a PC cladding, an optical fiber 110b may include a poly(carbonate) (PC) core with a poly(methyl methacrylate) (PMMA) cladding (n_{PC} = 1.586, n_{PMMA} = 1.49), and a microfluidic fiber 110c may have a microfluidic channel in a hollow PC fiber. Each type of fiber is drawn from a corresponding preform 11 with a furnace 2 heated to the appropriate temperature.

Once drawn, the fiber is cut into segments, and the ends of the segments are connected to the appropriate terminations. Each fiber can be connected at one end to an appropriate coupler or discrete component, such as a light source (optical fiber), electrical contact (electrical fiber), or fluid port (microfluidic fiber). For instance, an electrical fiber 110a can be connected to one or more electronic components 112a; an optical fiber 110b may be connected to one or more optical components 112b, such as fiber-coupled light source (e.g., a light source-emitting diode or laser) and/or photodetectors; and a microfluidic fiber 110c can be connected to backend fluidic tubing 112c that can be coupled to a pump or reservoir for delivery of an injectable compound as shown in FIG. 1B.

FIG. 1C shows a dipping and drying process called solvent evaporation or entrapment-driven (SEED) integration. In FIG. 1C, the process is carried out with three fibers—one electronic fiber 110a, one optical fiber 110b, and one microfluidic fiber 110c. More generally, SEED integration can be carried out with a single fiber, with two or more fibers of the same type (e.g., two microfluidic fibers 110c), or with fibers of different types, including types of fibers not shown in FIGS. 1A-1G, such as multifunctional fibers.

For SEED integration, the connectorized fibers are mounted on a motorized stage 4, shown in FIG. 1D, twisted (i) together into a fiber bundle 114, and secured (ii) e.g., using epoxy or a mechanical fastener 116. The fibers can also be secured in the fiber bundle 114 with epoxy or fasteners and without being twisted. The fiber bundle 114 is dipped (iii) in a poly(urethane)-poly(ethylene glycol) (PU-PEG) solution 123, then withdrawn from the PU-PEG solution 123 and heated (iv) to evaporate the solvent, leaving a layer of PU-PEG hydrogel 120 surrounding and securing the twisted fiber bundle 114. The coated fibers can then be cut (v) to the desired length, yielding the neural interface 100. If desired, once the PU-PEG layer is dry, the PU-PEG-coated fibers can be dipped in a hydrogel bath 131 (e.g., a hydrogel other than PU-PEG, such as collagen or a hydrogel that includes a protein, glycan, synthetic polymer, biopolymer, gelatin, laminin, hyaluronic acid, alginate, or Matrigel), then heated again to leave a (second) hydrogel layer 130 encapsulating the PU-PEG-coated fibers as shown in FIG. 1E before being cut to the desired length. Human breast cancer cells and human epithelial cells can interact with and penetrate the hydrogel layer, which can ultimately interface with the brain.

Other suitable fibers for neural interfaces include polymer-based multifunctional fibers. For example, FIG. 1F illustrates SEED integration of a polymer-based multifunctional fiber 110d. The polymer-based multifunctional fiber 110d has, within a single fiber form factor, embedded within it a combination of electrical, optical, and/or microfluidic modalities. (For more on multifunctional fibers, see, e.g., U.S. Pat. No. 9,861,810, which is incorporated herein by reference in its entirety for all purposes.) Its proximal end is connected to one or more electronic components 112a′, one or more optical components 112b′, and backend fluidic tubing 112c. The connectorized multifunctional fiber 110d is dipped into and withdrawn from a bath of PU-PEG (not shown), then dried to form a layer or coating of PU-PEG hydrogel 120′. If desired, the PU-PEG-coated multifunctional fiber 110d can be dipped in another bath of hydrogel, e.g., a bath or solution of collagen, protein, glycan, synthetic polymer, biopolymer, gelatin, laminin, hyaluronic acid, alginate, or Matrigel, to form an outer hydrogel layer or coating around the inner PU-PEG layer as shown in FIG. 1E. The multifunctional fiber 110d is then cut to the desired length to form a tissue-integrating neural interface 100″.

SEED integration can be used with other materials and can be repeated to create additional layers and/or thicker layers. For example, SEED integration can be extended from collagen hydrogel to almost any hydrogel system derived from proteins, glycans, synthetic polymers, and other materials where some hydrogen bonding is possible, such as gelatin, laminin, elastin-like protein, hyaluronic acid, alginate, Matrigel, etc. The hydrogen bonding with PU-PEG allows a consistent layer to be deposited. Materials which are commonly used for wound healing or other tissue engineering applications could be adapted to the neural interface’s hydrogel-based fiber system. SEED integration does not require any free radicals or other toxic byproducts and can accommodate a wide chemical toolbox previously inaccessible to multifunctional neural probes.

If desired, the hydrogel layer(s) can be loaded with molecules that interact or affect the brain, including proteins such as chemokines or cytokines, growth factors, angiogenic factors. Other suitable molecules for loading the hydrogel layer(s) include small molecules such as steroids, other small molecule drugs such as chemotherapies, neuromodulatory compounds, immunomodulatory compounds, chemotherapeutics, and/or other bioactive molecules.

The fibers can also be coated with cell-laden hydrogels. For example, the fibers can be integrated with stem cell-loaded hydrogels to enhance the wound healing process for tissue recovery. This can be done by dipping the fibers into a suspension of cells mixed with the hydrogel precursor solution. Other suitable cells include therapeutic cells, such as loading engineered T cells directly in the hydrogel fibers. The electrical, optical, and chemical modalities of the neural interface itself could be used to modulate these hydrogel-loaded cellular therapies.

### Experimental Demonstration of Tissue-Integrating Neural Interfaces

Employing a co-polymer of poly(ethylene glycol) tethered to water-insoluble poly(urethane) (PU-PEG), polymers with known biocompatibility routinely used in clinical implants and pharmaceuticals avoids sophisticated cleaning steps associated with potentially toxic radical initiators. Upon exposure to water, the PEG blocks facilitate hydration of the material while the hydrophobic forces between PU blocks prevent dissolution, resulting in a physical hydrogel. Since both blocks are soluble in ethanol, the co-polymer is dissolved in a 95% ethanol solution to form a PU-PEG bath. Bringing the individual fiber components together in this bath, and using a heat source to evaporate the solvent, results in an integrated assembly. This integration creates hydrogel fibers that maintain structural integrity upon insertion in a phantom brain model, and after implantation in vivo.

The fabricated hydrogel-integrated probes had excellent electrical, optical, and fluid delivery properties. The recording electrodes, with 25 µm tungsten wires, had an impedance of 80 kOhm at 1 kHz, which is well within the range suitable for extracellular recordings of neuronal potentials. Using tungsten instead of nickel chromium (NiCr) in the tetrodes avoids gold plating, which is used to achieve sub-MOhm impedance, as that could expose the hydrogel to an organic solvent. The 25 µm tungsten electrodes were selected over 12.5 µm tungsten electrodes because they had a lower impedance. Optical losses in the PC/PMMA waveguide were measured as 0.76 dB/cm loss at a 473 nm wavelength, which was consistent with previously observed losses in PC-core fibers and sufficient for optical neural excitation mediated by channelrhodopsin-2 (ChR2). The injection efficiency was >90% for injection rates above 10 nL/s, confirming efficient fluid delivery through the microfluidic channels. Finally, dynamic mechanical analysis (DMA) showed that the hydrogel neural interfaces were flexible, in particular compared to other commonly used devices in neuroscience.

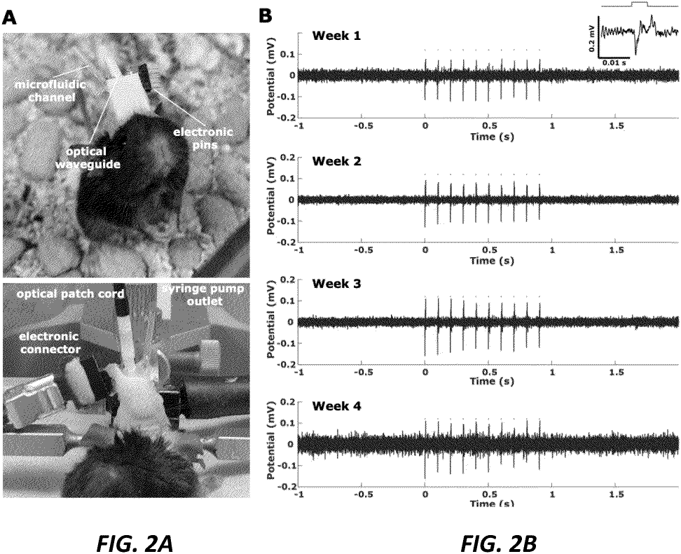

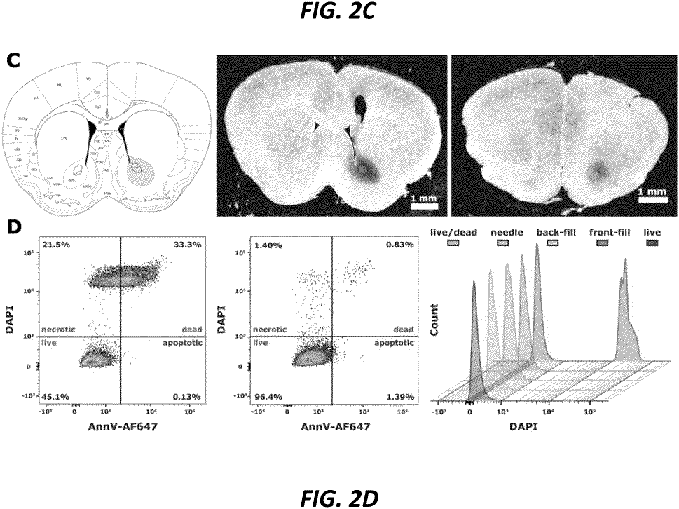

FIGS. 2A-2D illustrate the functionality of the fabricated hydrogel-based probes in transgenic mice broadly expressing ChR2 fused to a yellow fluorescent protein under the Thy1 promotor. ChR2 is a light-gated cation channel which, upon irradiation with blue light, causes neuronal depolarization and firing of action potentials. FIG. 2A shows a neural probe chronically implanted into the nucleus accumbens (NAc) of a Thy1-ChR2 mouse. The NAc plays an important role in the cognitive processing of reward and motivation, and its aberrant function has been implicated in a wide range of mental disorders, including schizophrenia, substance addiction, and post-traumatic stress disorder. The polymer-based optical waveguide and tungsten recording electrodes allowed optically stimulation and recording of simultaneously evoked activity, e.g., as shown in FIG. 2B for optical pulses at a wavelength of λ = 473 nm, 10 Hz pulse rate, 5 ms pulse width, and 20 mW/mm² intensity. The recording electrodes can also record spontaneous activity of NAc neurons in anesthetized mice. This activity was correlated with laser onset (jitter = 0.84 ms, mean peak latency = 5.86 ms). Electrophysiological recordings during optical stimulation in a chronically implanted Thy1-ChR2 mouse following euthanasia showed no evoked activity, indicating that the observed action potentials were not due to the photo-electrochemical (Becquerel) effect.

FIGS. 2C and 2D illustrate the neural interface’s microfluidic capabilities in vivo. They illustrate the delivery of Evans blue dye (2% in sterile saline) into the NAc of Thy1-ChR2 mice. Ten minutes following injection, the animals were transcardially perfused with 4% paraformaldehyde, and widefield microscopy of brain slices revealed a dye depot formation in the NAc. FIG. 2C shows a brain atlas image with the NAc highlighted by shading (left). A cross section (center) of a Thy1-ChR2 brain injected with 3 µL of Evans blue dye at 33 nL/s followed by fixation with 4% paraformaldehyde shows the location of the NAc bolus. A cross-section (right) of the same brain approximately 0.7 mm away shows the periphery of the depot. FIG. 2D shows a mixture of approximately 50% live and 50% dead or dying RAW-Blue murine macrophages (left) and cells injected through the microfluidic channel with the backfill method at 1 µL/min (middle). FIG. 2D (right) also shows similar viability when compared to injections with 26G NanoFil syringes and live cells left on ice. The histogram was normalized to the mode.

The results in FIGS. 2C and 2D show that inventive neural interfaces are compatible with cell-based therapies for understanding and treating neurological diseases—cells delivered through the integrated microfluidic channel of an inventive neural interface remain viable. Depending on the application, the therapeutic cargo can be front-filled into the tip of the microfluidic channel and delivered during device implantation surgery or can be back filled days or weeks following chronic implantation as shown in FIG. 2C.

RAW-Blue macrophages (RBMs) remain viable using both delivery strategies as verified by flow cytometric analysis with DAPI and Annexin V (AnnV) conjugated to Alexa Fluor 647 (AnnV-AF647). As a DNA-binding dye, DAPI was used to probe the viability of cells as fluorescence is only observed when cell membrane integrity is lost during cell death. Annexin V was used to identify exposed aminophospholipid phosphatidylserine (PS). PS is normally maintained on the inner leaflet of the cell membrane under physiological conditions but becomes exposed during the early stages of regulated cell death and serves as a phagocytic signal. Together, these markers enable quantification of apoptotic and necrotic processes in response to cell stresses or treatments. We applied these markers to compare viability of cells delivered through the microfluidic channel within the hydrogel neural probe to those kept on ice, injected with a 26G NanoFil syringe, or killed via heat shock, with results shown in FIG. 2D. With either the front- or back-fill approach, the neural interfaces retained RBM cell viability >90% at a 1 µL/min injection rate, suggesting that these probes can be used to deliver live cells directly into the central nervous system.

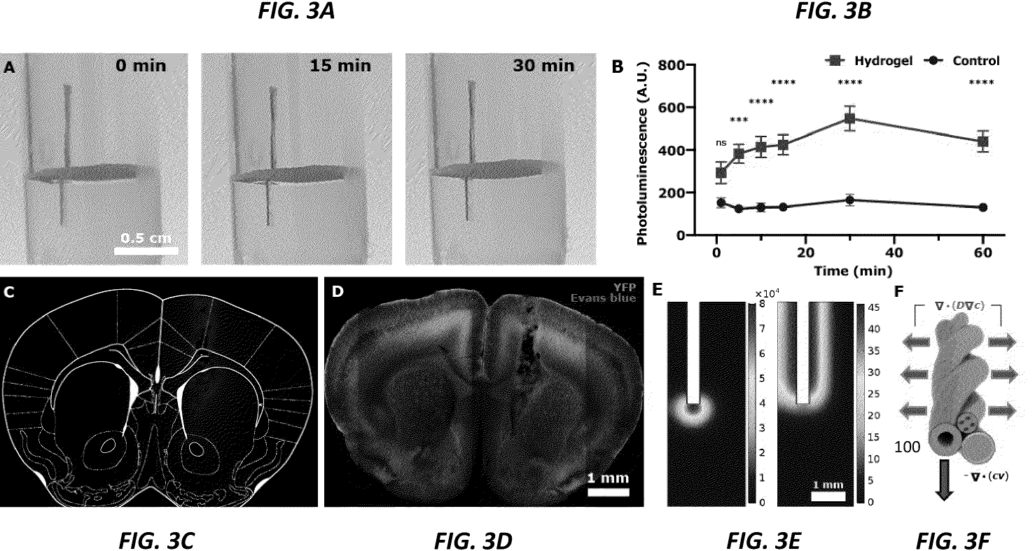

FIGS. 3A-3F illustrate how an inventive neural interface’s hydrogel cladding enables delivery of small molecules along the entire length of the fiber portion of the neural interface. In other words, FIGS. 3A-3F illustrate drug delivery of molecules loaded into the hydrogel itself, independent of the microfluidic channel. We used fluorescein as a model drug and co-loaded it into the hydrogel precursor PU-PEG ethanol solution. Since fluorescein is water soluble, introduction into an agarose phantom brain results in diffusion of this molecule away from the hydrogel as shown in FIG. 3A.

FIG. 3B is a plot of photoluminescence emitted fluorescein released from integrated fibers with and without PU-PEG (upper and lower traces, respectively) versus time. The nonhydrogel control condition (lower trace) was the SEED integration with an equivalent concentration of fluorescein without hydrogel. In addition to depositing a lower concentration, the control integrated fiber did not stay integrated without the hydrogel in PBS. Statistical analysis was conducted using two-way ANOVA, with n = 4 in each group, ***P = 0.0002, ****P < 0.0001. The error bars in FIG. 3B are ± s.e.m. This quantitative analysis of release into PBS shows a bolus release that peaks 30 minutes post-insertion.

FIGS. 3C and 3D show a demonstration this neural interface capability in vivo by co-loading Evans blue into the hydrogel of neural interfaces and implanting the neural interfaces into the NAc of Thy1-ChR2 mice. After 72 hours, the mice were perfused and brain slices were taken. FIGS. 3C and 3D show confocal microscopy images of these brain slices (4× objective, scans of regions stitched with FluoView software package). These confocal microscopy images reveal that hydrogel-loaded Evans blue has a different in vivo release profile compared to delivery through the microfluidic channel.

FIGS. 3E and 3F illustrate mechanisms for Evans blue delivery via the neural interface’s microfluidic channel and via its hydrogel layer. FIG. 3E shows plots of finite element modeling of mass transport of a small molecule, such as Evans blue, from either a microfluidic channel (left) or hydrogel (right) of a tissue-integrating neural interface 10 minutes after completion of the 3 µL injection (same time point in both cases). FIG. 3F illustrates both microfluidic and hydrogel-based drug delivery and the resulting convection versus diffusion driving forces, respectively.

Instead of convection-driven transport (first term on the righthand side in Eq. 1, below) at the tip of the neural interface, Evans blue delivery is dominated by diffusion-driven transport (second term on the righthand side in Eq. 1) and happens along the whole length of the implant. This additional drug delivery modality enabled by the hydrogel may be more advantageous for certain applications, such as the modulation of the foreign body response using anti-fibrotic drugs eluted along the length of implants.

$$\frac{\partial c}{\partial t} = - ∇ ⋅ \left(c v\right) + ∇ ⋅ \left(D ∇ c\right) + R \qquad \text{(1)}$$

Controlled delivery of hydrophobic small-molecule drugs remains a formidable obstacle to the translational utility of small-molecule drugs. Despite recent setbacks, emergent clinical applications of hydrophobic molecules, such as cannabinoids, have garnered renewed interest in their effective delivery. Rationally designed polymers can overcome the delivery challenges of hydrophobic small molecules by, for example, forming nanodroplets that can carry these molecules into the cytosol. These custom polymers are melts at room temperature, with glass transition temperatures > 150° C. lower than that of PU-PEG, and are not co-drawable with structural polymers typically used in fiber drawing of neural probes. SEED integration allows us to overcome these challenges and thus expands the drug delivery capabilities of neural interfaces.

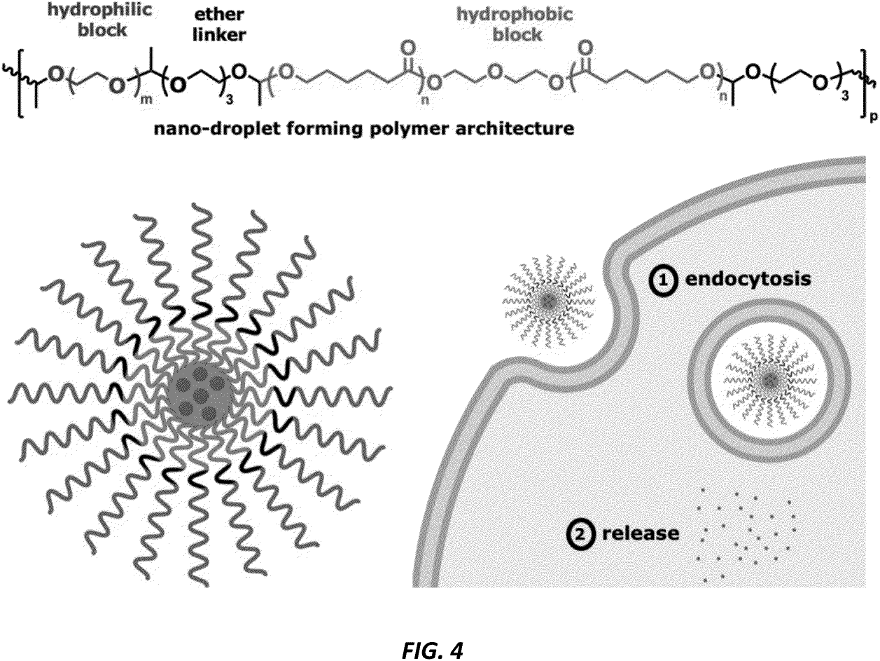

FIG. 4 illustrates delivery of hydrophobic compounds with a custom block co-polymer of PEG and poly(caprolactone) (PCL) synthesized with an acid-labile ether linkage. This poly(acetal), named PA11 (1:1 ratio of PEG:PCL), self-emulsifies into nanodroplets capable of delivering hydrophobic small-molecule drugs as shown at bottom left of FIG. 4. The center of the nanodroplet in FIG. 4 is shaded to illustrate the drug-loading capability in the hydrophobic region of the polymer. The bottom right portion of FIG. 4 shows endosomal escape of the hydrophobic small-molecule drug.

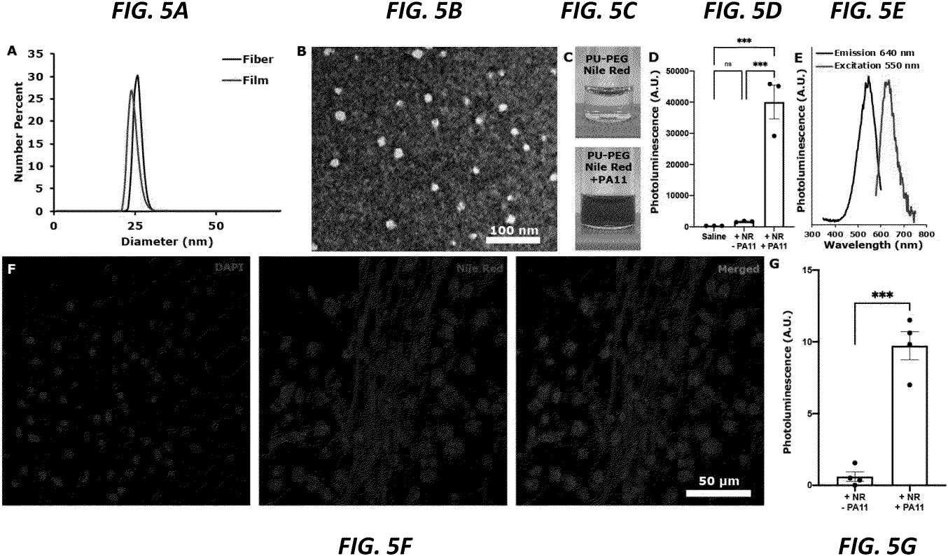

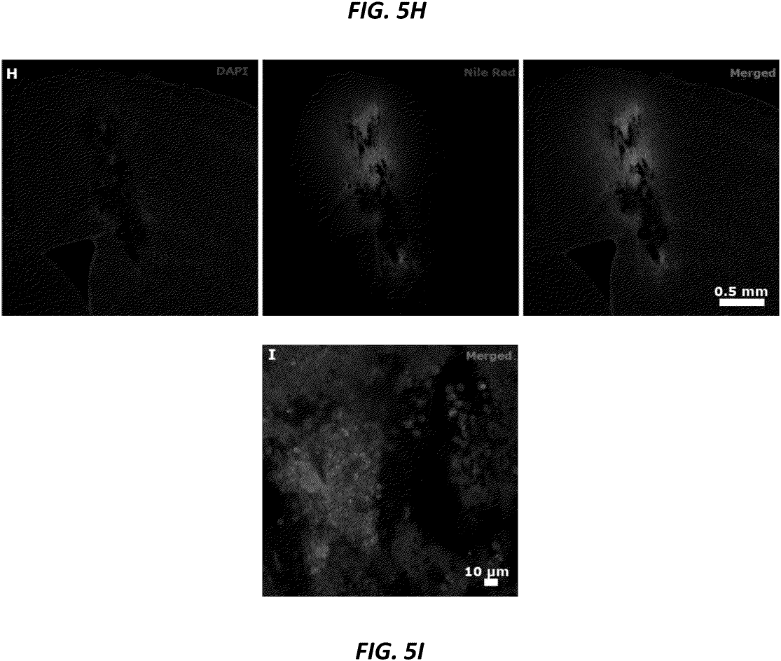

FIGS. 5A-5I illustrate results of experiments performed with PA11 that was blended with a PU-PEG precursor solution at a 3:17 ratio, with the resulting blend applied to a fiber assembly during SEED integration of a tissue-integrating neural interface. PA11 releases from the PU-PEG matrix and forms nanodroplets approximately 25 nm in diameter in saline solution under mild perturbations. As a positive control, we repeated the experiment with a thick film of the PU-PEG/PA11 blend and vigorously shook it overnight, which resulted in PA11 nanodroplets of similar size.

FIG. 5A shows dynamic light scattering (DLS) data of the solution after elution of PA11 from PA11/PU-PEG-based fibers/neural interfaces (right peak) and from the control PA11/PU-PEG film (left peak). The DLS data indicate similar PA11 nanodroplet diameters from the fibers and film. FIG. 5B shows transmission electron microscopy (TEM) images of the dehydrated samples. These images corroborate the nanodroplet dimensions from the DLS data.

FIGS. 5C-5I illustrate the ability of PA11 to deliver hydrophobic compounds into aqueous media. When the hydrophobic small molecule Nile Red was mixed in with the PA11:PU-PEG composite, PA11 could escape and carry the hydrophobic dye with it as shown in FIG. 5C. FIGS. 5D and 5E show immersing Nile Red-loaded hydrogel fibers with and without PA11 into PBS and measuring the fluorescence after gentle shaking overnight, the hydrophobic small molecule is released only when PA11 was co-loaded into the hydrogel. More specifically, FIG. 5D shows quantification via fluorescence spectroscopy of Nile Red loaded into hydrogel neural interfaces with and without PA11. Statistical analysis was conducted using ordinary one-way ANOVA. n = 3 in each group, ***P < 0.001 (P = 0.0003 vs saline, P = 0.0004 vs PA11). The error bars in FIG. 5D are ± s.e.m. The excitation and emission spectra (left and right, respectively) shown in FIG. 5E were obtained with a fixed emission wavelength of 640 nm and a fixed excitation wavelength of 550 nm, respectively.

FIG. 5F shows images of primary rat dorsal root ganglion (DRGs; sensory neuronal structures) incubated with Nile Red in the presence or absence of PA11. FIG. 5F shows that after a 24-hour incubation with PA11, 96% of neurons were Nile Red-positive. FIG. 5G shows the quantification of intensity on the Nile Red channel compared to no PA11 control. Statistical analysis was conducted using unpaired t test. n = 4 in each group, ***P = 0.0001. No dye was found in neuronal cytoplasm or nuclei in the absence of PA11, suggesting that PA11 is sufficient for effective intra-neuronal delivery of hydrophobic small molecule drugs. FIGS. 5H and 5I are images of an in vivo demonstration of NR/PA11 elution from a neural interface in the NAc of C57BL/6 mice after 72 hours at 4× and 60× magnification, respectively.

### Optical Fibers for Neural Interfaces

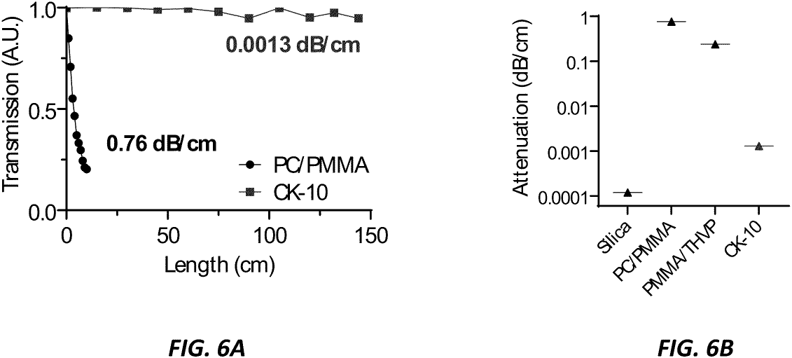

FIGS. 6A and 6B illustrate the optical performance of a CK-10 polymer optical waveguide used as an optical fiber in a tissue-integrated neural implant for neurobiology and cancer. FIG. 6A shows the optical loss versus fiber length for CK-10 (lower trace) optical fiber, which has a fluorinated polymer cladding around a PMMA core, and for an optical fiber with PC core and PMMA cladding (upper trace). The CK-10 optical fiber has significantly lower loss. FIG. 6B shows optical attenuation for different silica, PC/PMMA, PMMA/THVP, and CK-10 optical waveguides materials in multifunctional fibers. Silica optical fiber has lower attenuation but is less flexible and therefore less suitable for implantation than CK-10 optical fiber. The CK-10 optical fiber’s low less and flexibility make it well-suited for use in a multifunctional, tissue-integrating neural interface that is deployed to interface with the nervous system and cancer in the brain and the periphery.

### Carbon Nanotube Electrical Fibers for Neural Interfaces

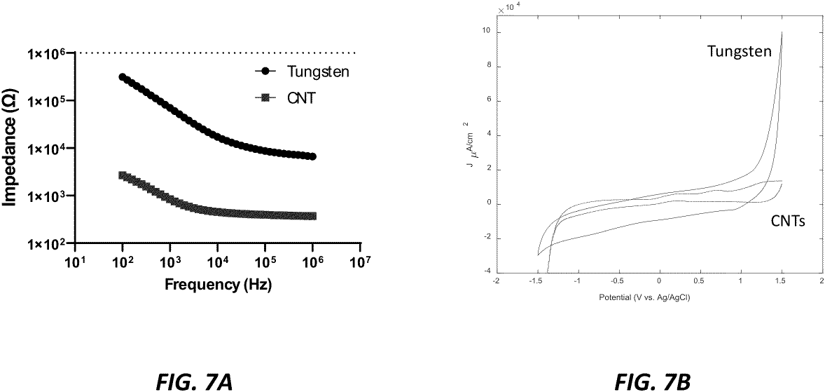

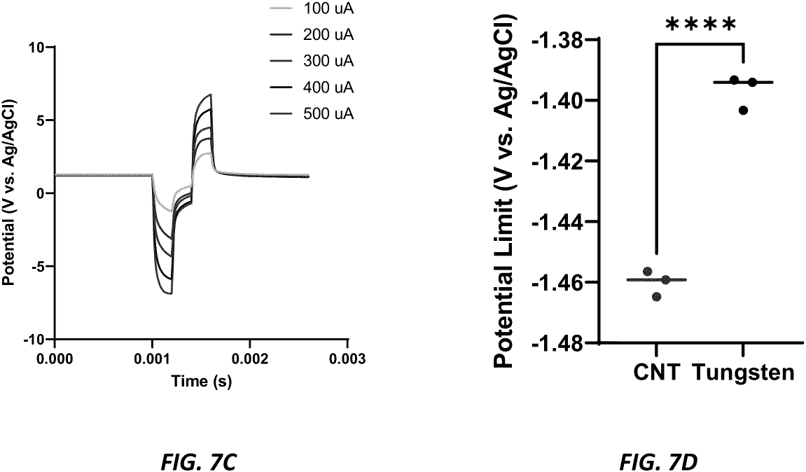

FIGS. 7A-7D illustrate the performance on electrical fibers with carbon nanotube (CNT) and tungsten conductors in a hydrogel neural interface. Carbon nanotubes within a hydrogel fiber-based neural interface can be used to stimulate cells in the brain and periphery. FIG. 7A shows the impedance versus frequency of tungsten (upper trace) and CNT (lower trace) electrical fibers. FIG. 7B shows current-voltage (IV) curves for CNTs and tungsten wires. FIGS. 7C and 7D show the potential relative to a reference potential for CNTs and the potential limit for CNTs versus tungsten, respectively, illustrating that CNTs in these devices are well posed to inject current. Generally, any metal or conductor should perform well and maintain its properties in a hydrogel neural interface.

### Optical and Electrical Stimulation and Recording With Implanted Neural Interfaces

Neural interfaces coated hydrogel can be used for optical stimulation and recording. They can interface with the peripheral nervous system and adhere well to peripheral tissues. They can be used for detecting cancer cells and for optically and/or electrically stimulating and recording tumors.

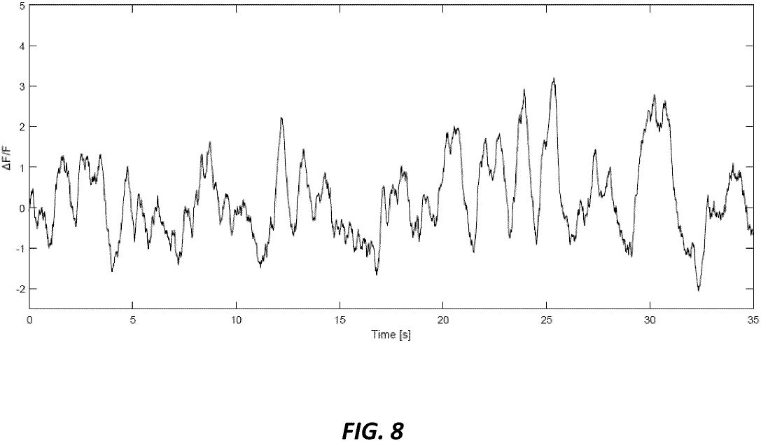

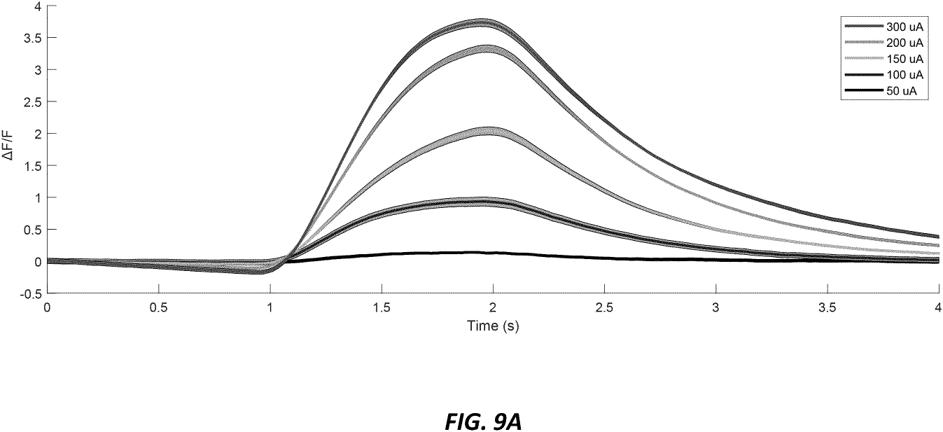

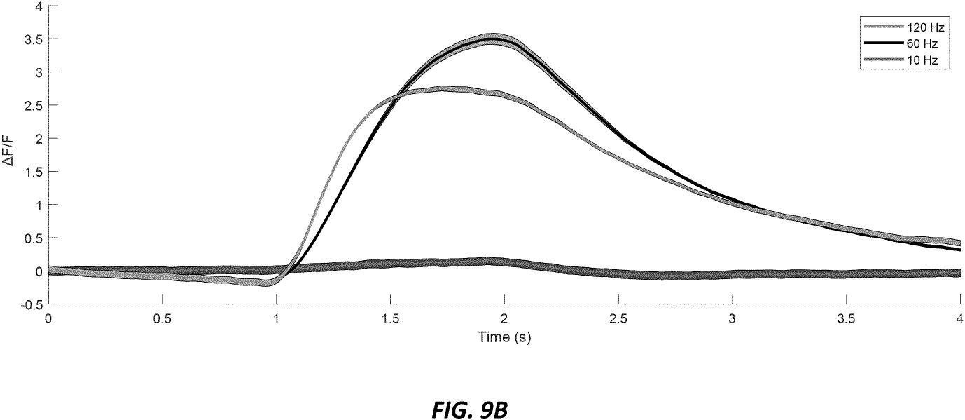

FIGS. 8, 9A, and 9B show that a CK-10 optical waveguide and CNT electrodes within a hydrogel neural interface can optically stimulate neurons, electrically stimulate neurons, optically record from neurons, optically record from cancer cells, and electrically stimulate cancer cells. FIG. 8 illustrates endogenous activity in the form of change in normalized fluorescence over time from neurons expressing GCaMP. This in vivo neural activity was evoked optically and recorded with an implanted hydrogel neural interface with a CK10 optical fiber.

FIGS. 9A and 9B show optical recordings of electrically evoked activity (fluorescence) from neurons expressing GCaMP. This activity was evoked and recorded with an implanted hydrogel neural interface with a CNT electrical fiber that injected alternating current (AC) electrical signals into the tissue with different current levels (FIG. 9A) and frequencies (FIG. 9B). Both plots show that the magnitude of the fluorescence is a function of the injected current. In both cases, electrical stimulus began at 1 second. Together, FIGS. 9A and 9B demonstrate simultaneous use of optical and conductive traces in an implanted neural interface.

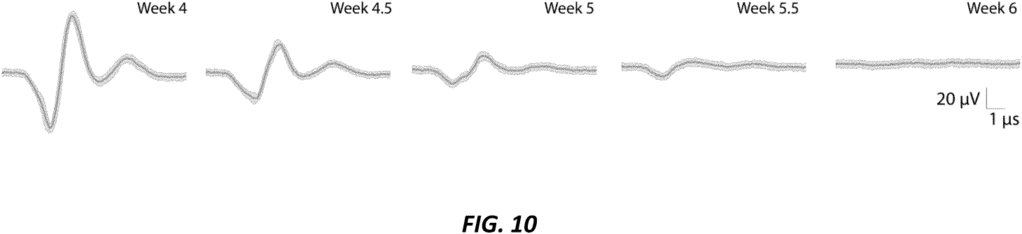

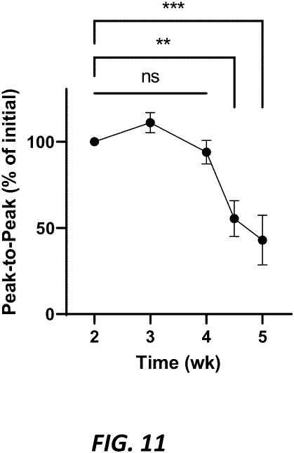

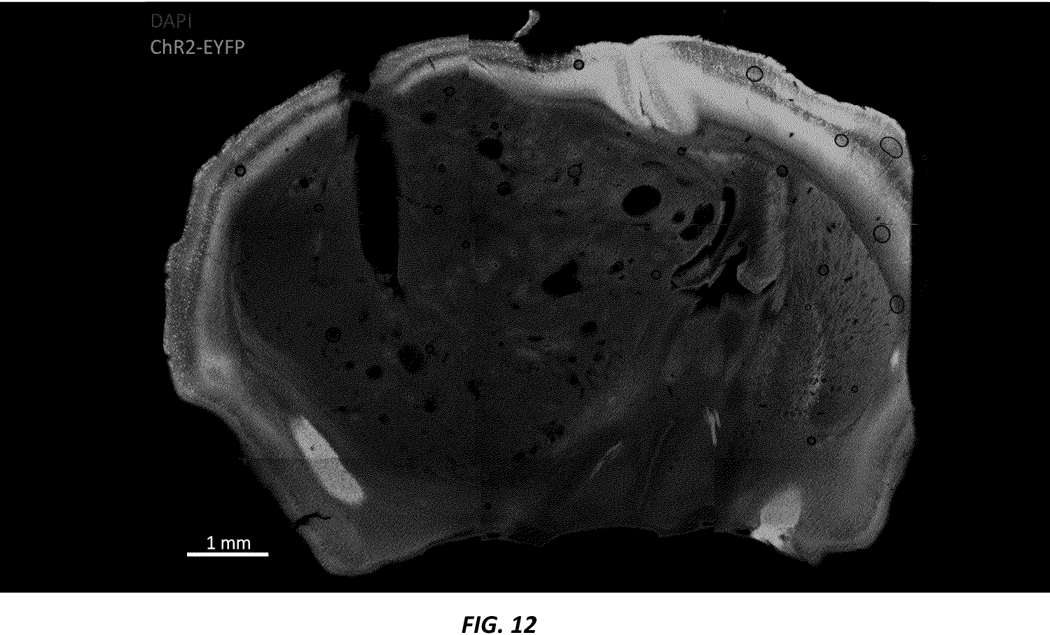

FIG. 10 shows traces of electrical activity evoked by optically stimulating neurons expressing ChR2. These traces were obtained over six weeks with a hydrogel neural interface chronically implanted near a brain tumor in a live mouse. The neurons were stimulated with light guided by an optical fiber in the hydrogel neural interface, and the electrical activity was recorded with tungsten electrodes in the hydrogel neural interface. This is done near a tumor, illustrating that they can be used to manipulate and record neuronal activity in the context of brain cancer. FIG. 11 shows a quantification over several channels of the peak-to-peak distance in the traces shown in FIG. 10.

FIG. 12 shows a section of a mouse brain after removal of a chronically implanted hydrogel neural interface. The lighter regions at upper right, lower left, and lower right are regions of cancer cells. The other regions are regions of normal tissue. And the void at upper left indicated where the neural interface was implanted. The size and shape of the void indicates that the neural interface did not migrate as the tumor grew.

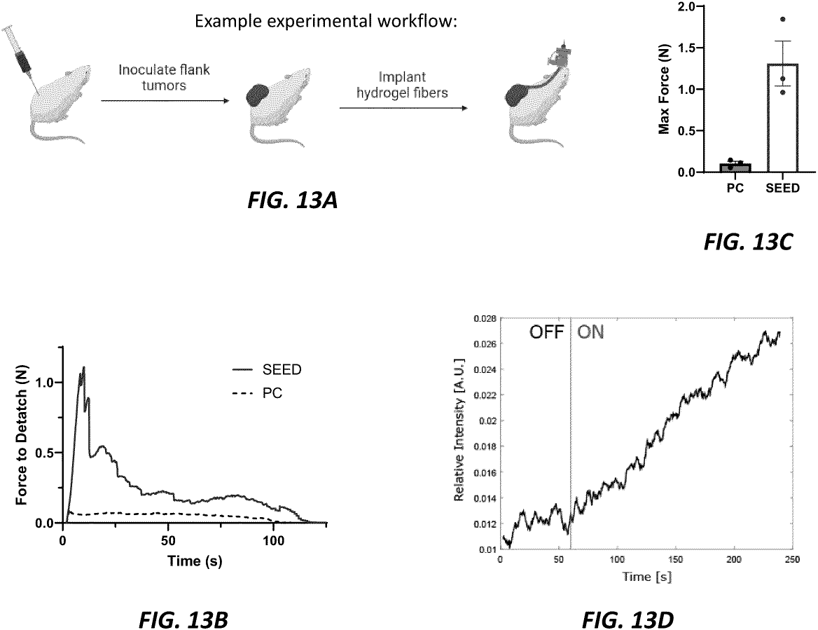

FIGS. 13A-13D illustrate how a hydrogel neural interface can be adhered to peripheral tissue, then used to electrically stimulate cancer cells while simultaneously recording their electrical and/or optical activity. FIG. 13A illustrates a process for implanting the fibers, with implantation preceded by inoculation of the tumor. FIGS. 13B and 13C show data indicating how well a neural interface adheres to the tissue. FIG. 13B shows the force versus time required to detach both a (SEED integrated) hydrogel neural interface (solid trace) and a conventional polymer fiber (dashed trace) stuck to skin using a bioadhesive. FIG. 13C shows the maximum detachment forces for hydrogel neural interfaces and conventional polymer fibers. It takes about ten times more force to detach the hydrogel neural interfaces than the conventional polymer fibers. And FIG. 13D shows electrical disruption of melanoma in the flank of a mouse by an implanted neural interface that simultaneously records calcium activity (fluorescence) in vivo.

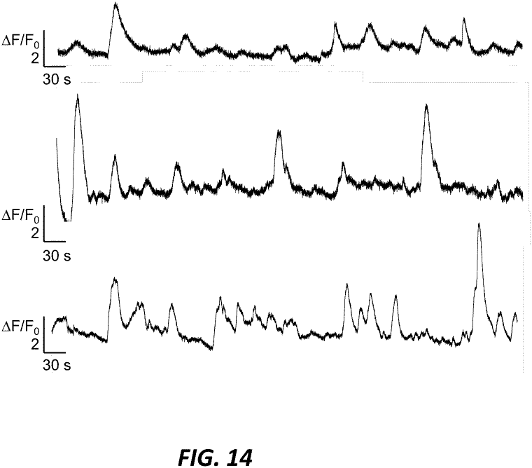

FIG. 14 shows traces of intracellular calcium transients detected by a fluorescence calcium indicator from cancer cells recorded in vivo by chronically implanted hydrogel neural interfaces.

### Drug Delivery With Implanted Neural Interfaces

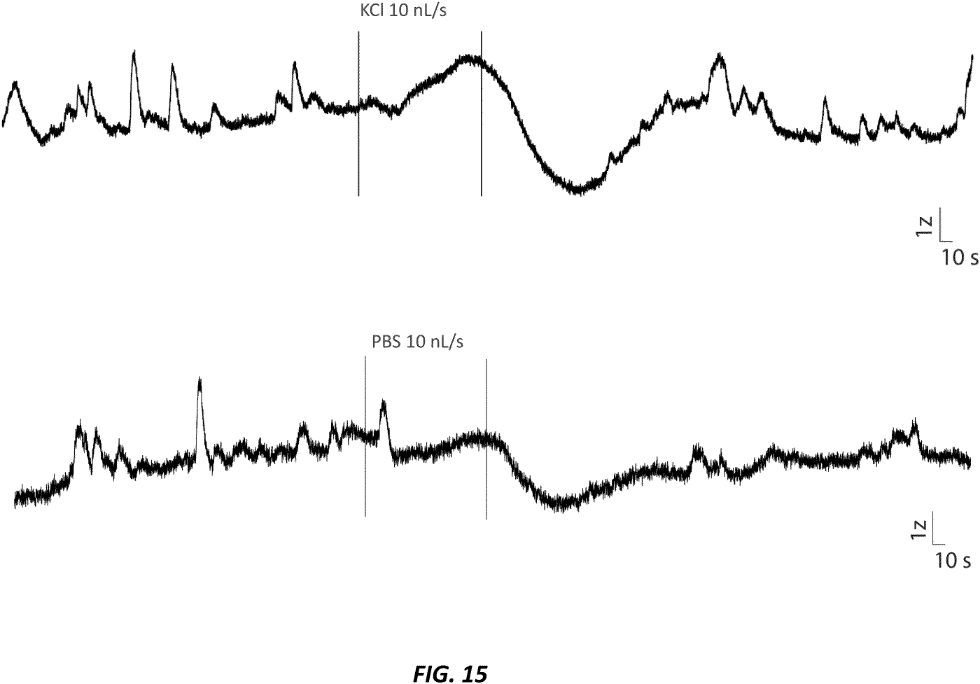

FIG. 15 shows fluorescence versus time measured with a CK10 optical fiber in an implanted neural interface while the implanted neural interface’s microfluidic fiber deliver different solutions to the surrounding tissue. The upper trace shows change in fluorescence for a KCl solution, and the lower shows change in fluorescence for a PBS solution. These plots show that a neural interface can be used to inject solutions directly into a tumor chronically in vivo. Suitable solutions include small-molecule chemotherapies, immunotherapies, other drugs, imaging agents, radio therapy agents, or anything else that can be formulated into an injectable solution.

### Conclusion

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain, using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the U.S. Pat. Office Manual of Patent Examining Procedures, Section 2111.03.

1. A method of making a neural interface, the method comprising:
forming a fiber bundle from a plurality of fibers;
at least partially coating the fiber bundle in a layer of poly(urethane)-poly(ethylene glycol) (PU-PEG); and
at least partially coating the layer of PU-PEG in a layer of hydrogel.

2. The method of claim 1, wherein the plurality of fibers comprises at least one of an optical fiber, an electrical fiber, or a microfluidic fiber.

3. The method of claim 1, wherein the plurality of fibers comprises an optical fiber, an electrical fiber, and a microfluidic fiber.

4. The method of claim 1, wherein at least partially coating the fiber bundle in the layer of PU-PEG comprises dipping the fiber bundle in a solution of PU-PEG and drying the solution of PU-PEG on the fiber bundle.

5. The method of claim 1, wherein at least partially coating the layer of PU-PEG in the layer of hydrogel comprises dipping the fiber bundle in a hydrogel bath after forming the layer of PU-PEG on the fiber bundle.

6. The method of claim 1, wherein the layer of hydrogel comprises at least one of a protein, glycan, synthetic polymer, biopolymer, gelatin, laminin, hyaluronic acid, alginate, or Matrigel.

7. The method of claim 1, further comprising:
loading the layer of hydrogel with a molecule configured to interact with and/or affect a human brain.

8. The method of claim 1, further comprising:
loading the layer of hydrogel with at least one of a hydrophobic molecule, a hydrophilic molecule, a peptide, or a protein.

9. The method of claim 1, further comprising:
loading the layer of hydrogel with cells.

10. A neural interface comprising:
a fiber bundle comprising a plurality of fibers;
a layer of poly(urethane)-poly(ethylene glycol) (PU-PEG) at least partially surrounding the fiber bundle; and
a layer of hydrogel at least partially surrounding the layer of PU-PEG.

11. The neural interface of claim 10, wherein the plurality of fibers comprises at least one of an optical fiber, an electrical fiber, or a microfluidic fiber.

12. The neural interface of claim 10, wherein the plurality of fibers comprises an optical fiber, an electrical fiber, and a microfluidic fiber.

13. The neural interface of claim 10, wherein the layer of hydrogel comprises at least one of a protein, glycan, synthetic polymer, biopolymer, gelatin, laminin, hyaluronic acid, alginate, or Matrigel.

14. The neural interface of claim 10, wherein the layer of hydrogel comprises collagen.

15. The neural interface of claim 10, wherein the layer of hydrogel is loaded with a molecule configured to interact with and/or affect a human brain.

16. The neural interface of claim 10, wherein the layer of hydrogel is loaded with at least one of a hydrophobic molecule, a hydrophilic molecule, a peptide, a protein, or a virus.

17. The neural interface of claim 10, wherein the layer of hydrogel comprises a cell-laden hydrogel.

18. A method of making a neural interface, the method comprising:
dipping a fiber into a solution of poly(urethane)-poly(ethylene glycol) (PU-PEG);
withdrawing the fiber from the solution of PU-PEG; and
drying the solution of PU-PEG on the fiber to form a PU-PEG coating on the fiber.

19. The method of claim 18, wherein the fiber is a multifunctional fiber.

20. The method of claim 18, further comprising:
after drying the solution of PU-PEG on the fiber, dipping the fiber in a hydrogel solution;
withdrawing the fiber from the hydrogel solution; and
drying the hydrogel solution on the fiber to form a hydrogel coating on the PU-PEG coating.
